(19) United States
(12) Patent Application Publication
(10) Pub. No.: US 2010/0297691 A1
(43) Pub. Date: Nov. 25, 2010

(54) DEVICES AND PROCESSES FOR COLLECTING AND CONCENTRATING SAMPLES FOR MICROBIOLOGICAL ANALYSIS

(76) Inventors: Alice Maria M. Ribeiro, Sao Paulo (BR); Cristina F. Abreu, Sao Paulo (BR); Claudia R. Kashiwakura, Sao Paulo (BR); Carlos B. Junior, Sao Paulo (BR); Adriana R. Tassinari, Sao Paulo (BR); Daniel R. Mclntyre, St. Paul, MN (US); Michael G. Williams, Vadnais Heights, MN (US)

Correspondence Address: 3M INNOVATIVE PROPERTIES COMPANY, PO BOX 33427, ST. PAUL, MN, 55133-3427, US

(21) Appl. No.: 12/601,278

(22) PCT Filed: May 28, 2008
(86) PCT No.: PCT/US08/64939

§ 371 Date: Aug. 5, 2010

Related U.S. Application Data
(60) Provisional application No. 60/941,145, filed on May 31, 2007.
(60) Provisional application No. 60/941,150, filed on May 31, 2007.

(51) Int. Cl. C12Q 1/04; C12M 1/34

(52) U.S. Cl. 435/34; 435/287.1

(57) ABSTRACT

The present invention refers to manual devices for the collecting and concentrating liquid samples for microbiological analysis and their respective methods of use. The manual devices comprise a body which contains a sample, a removable support, a microporous membrane, and a plunger. The present invention is also directed to methods of collecting and concentrating of liquid samples onto a microporous membrane for micro-biological analysis.

### CROSS REFERENCE TO RELATED APPLICATIONS

This application claims the benefit of U.S. Provisional Patent Application Nos. 60/941,145 and 60/941,150; each filed on May 31, 2007, each incorporated herein by reference in its entirety.

### BACKGROUND

In many countries, the quality of water for human consumption is determined by parameters established in regulations or standards, which define the acceptable limits of contaminants such as, for example, organic and biological material, in order for the water to be considered drinkable (i.e. appropriate for human consumption). Microbiological standards, in which the determination of the absence or presence of coliform bacteria is a basic analysis, determine the sanitary quality of water that is used for many purposes. The quality of food for human consumption is also regulated in certain countries by norms or standards, which define acceptable limits of microbial contaminants, such as total aerobic bacteria, coliform bacteria, yeast, and mold. The presence of certain microorganisms in food or water can create significant health risks to the individuals or communities that consume the contaminated food or beverages.

The presence of coliform bacteria is an important indication of food and water quality. The permitted amount of coliform bacteria found in drinking water or certain foods, such as dairy products, is regulated in many countries and/or municipalities. Coliforms are gram-negative, oxidase negative, facultative aerobic bacteria, which do not form spores, are capable of growing in the presence of bile salts or tensoactive agents, and which ferment lactose with the production of acid, gas and aldehyde. Among the group of coliforms, there is a specific group, the fecal coliforms, whose main representative is Escherichia coli. The presence of fecal coliforms in a sample is a primary indication of recent fecal contamination of the sample water, and of the possible presence of pathogenic organisms.

Methods for enumerating microbes in water samples can be found in, for example, the compendium “Standard Methods for the Examination of Water and Wastewater” (SMEWW), 21^{st} Edition, which is a joint publication of the American Public Health Association, the American Water Works Association, and the Water Environment Federation. SMEWW describes a membrane filtration technique to obtain a direct count of microorganisms in samples containing a large volume of water. This technique is reproducible and can generally produce numeric results more quickly than alternative procedures that involve fermentation in multiple tubes of broth medium containing a specific carbohydrate. The membrane filtration technique is useful in monitoring the microbiological quality of samples from processes intended to produce drinking water, as well as samples from a variety of natural, unprocessed water sources.

Methods for enumerating microbes in food samples often vary according to the nature of the food and the types of organisms that are likely to be found in the samples. Several compendia of methods for testing food samples include “Standard Methods for the Examination of Dairy Products”, 27^{th} Edition, published by The American Public Health Association, Washington, D.C., the Bacteriological Analytical Manual (“BAM”), published by the U.S. Food and Drug Administration, Washington, D.C., and “The Encyclopedia of Food Microbiology”, published by Elsevier, Inc., Burlington, Mass. Solid foods are often suspended in aqueous media, such as Standard Methods Buffer, and mixed and/or pulverized to obtain a liquid homogenate of the food material. The liquid homogenates provide relatively uniform suspensions of the food sample and its microbial flora, and the homogenates are useful for several methods of quantitative microbial analysis.

Devices and processes have been developed to facilitate the concentration of microorganisms in water samples collected in the field. Typically, these samples are transported to a microbiology laboratory, where they can be analyzed to determine the number and identity of microorganisms that were present in the sample. One such device, described in U.S. Pat. No. 4,871,662, comprises a stabilizing agent to maintain the viability of the microorganisms during transport of the device from the field testing site to the laboratory.

Millipore Corporation (Billerica, Mass.) markets a filter holder under the trade name SWINNEX®. A microporous membrane can be placed inside a SWINNEX filter holder and, after sterilization, the device can be connected to a syringe or a tube to pass a solution through the filter to effect disinfection or sterilization of the solution. Millipore Corporation also manufactures glass filter holders that consist of a housing with volumetric indicia, a base with a porous filter support, and a spring clamp to attach the housing to the base. The glass filter holders are used with sterile microporous membrane filter to remove particulate material, including bacteria, from a liquid sample. The filter holders are connected to a vacuum source to pull the liquid sample through the membrane filter.

The devices for the microbiological analysis of water samples currently available are generally expensive, requiring various stages for analysis and a sophisticated laboratory infrastructure, as well as highly trained personnel for the manipulation of the material.

Some of the devices currently available are operated using vacuum pumps and/or complex filtration devices, some of which involve manifolds and/or more than one filter to concentrate the microorganisms in the sample. These devices add to the complexity and length of the operation, and require the availability of trained personnel to perform the analyses, resulting in increased costs. Furthermore, some of the devices require one or more transfer of the samples from one location or receptacle to another, which increases the risk of contamination of the samples with microorganisms that were not present in the original sample.

Accordingly, there is a technical need for a simplified device which allows the collection and concentration of liquid samples to be carried out in a single step. Furthermore, there is a technical need for a device that provides for liquid sample acquisition and filtration in efficient, simple, and economic processes.

### SUMMARY

The present invention relates to devices and kits for collecting and processing liquid samples for microbiological analysis. The present invention also relates to processes for collecting and concentrating liquid samples for microbiological analysis using the abovementioned manual devices and kits.

In one aspect, the present invention provides a device for collecting and concentrating a sample for microbiological analysis. In these embodiments, the device comprises a plunger dimensioned to provide an essentially watertight fit within a body, a removable support configured to position a microporous membrane in a flow path, and a body comprising a chamber wall and a base configured to attach to the removable support. The removable support comprises a porous support structure and a drain. In these embodiments, the body, the porous support structure, and the drain define a flow path for a liquid sample. In these embodiments, the device is configured to hold a predetermined volume of liquid sample, which is bounded at least in part by the removable support configured to position a microporous membrane in a flow path.

In another aspect, the present invention provides a device for collecting and concentrating a sample for microbiological analysis. In these embodiments, the device comprises a plunger dimensioned to provide an essentially watertight fit within a body, a microporous membrane, a removable support configured to position a microporous membrane in a flow path, and a body comprising a chamber wall and a base configured to attach to the removable support. The removable support comprises a porous support structure and a drain. In these embodiments, the body, the microporous membrane, the porous support structure, and the drain define a flow path for a liquid sample. In these embodiments, the device is configured to hold a predetermined volume of liquid sample, which is bounded at least in part by the removable support configured to position a microporous membrane in a flow path.

In another aspect, the present invention provides a device for collecting and concentrating a sample for microbiological analysis. In these embodiments, the device comprises a plunger dimensioned to provide an essentially watertight fit within a body, a microporous membrane, a removable support configured to position a microporous membrane in a flow path, and a body comprising a chamber wall and a base configured to attach to the removable support. The removable support comprises a porous support structure and a drain. In these embodiments, the body, the microporous membrane, the porous support structure, and the drain define a flow path for a liquid sample. In these embodiments, the device is configured to hold a predetermined volume of liquid sample, which is bounded at least in part by the removable support configured to position a microporous membrane in a flow path and is further defined by a fill line indicium.

In another aspect, the present invention provides a device for collecting and concentrating a sample for microbiological analysis. In these embodiments, the device comprises a plunger dimensioned to provide an essentially watertight fit within a body, a removable support configured to position a microporous membrane in a flow path, and a body comprising a chamber wall, a base configured to attach to the removable support, a plunger orifice, and a sample intake port. The removable support comprises a porous support structure and a drain. In these embodiments, the sample intake port, the body, the porous support structure, and the drain define a flow path for a liquid sample and the device is configured to hold a predetermined volume of liquid sample.

In another aspect, the present invention provides a device for collecting and concentrating a sample for microbiological analysis. In these embodiments, the device comprises a plunger dimensioned to provide an essentially watertight fit within a body, a removable support configured to position a microporous membrane in a flow path, a valve, and a body comprising a chamber wall, a base configured to attach to the removable support, a plunger orifice, and a sample intake port. The removable support comprises a porous support structure and a drain. In these embodiments, the valve, the sample intake port, the body, the porous support structure, and the drain define a flow path for a liquid sample and the device is configured to hold a predetermined volume of liquid sample.

In another aspect, the present invention provides a device for collecting and concentrating a sample for microbiological analysis. In these embodiments, the device comprises a plunger dimensioned to provide an essentially watertight fit within a body, a microporous membrane, a removable support configured to position a microporous membrane in a flow path, a valve, and a body comprising a chamber wall, a base configured to attach to the removable support, a plunger orifice, and a sample intake port. The removable support comprises a porous support structure and a drain. In these embodiments, the valve, the sample intake port, the body, the microporous membrane, the porous support structure, and the drain define a flow path for a liquid sample and the device is configured to hold a predetermined volume of liquid sample.

In another aspect, the invention provides a process for collecting and concentrating liquid samples for microbiological analysis, the process comprising providing a liquid sample to be analyzed, providing a device for collecting and concentrating samples for microbiological analysis, transferring the liquid sample to the interior of the device without the use of an intermediate sample collector, and applying force to the plunger to urge the liquid sample through a microporous membrane. In these embodiments, the device comprises a plunger dimensioned to provide an essentially watertight fit within a body, a microporous membrane, a removable support configured to position a microporous membrane in a flow path, and a body comprising a chamber wall and a base configured to attach to the removable support. The removable support comprises a porous support structure and a drain. In these embodiments, the body, the microporous membrane, the porous support structure, and the drain define a flow path for a liquid sample. In these embodiments, the device is configured to hold a predetermined volume of liquid sample, which is bounded at least in part by the removable support configured to position a microporous membrane in a flow path.

In another aspect, the invention provides a process for enumerating microorganisms in a sample comprising providing a liquid sample to be analyzed, providing a device for collecting and concentrating a sample for microbiological analysis, transferring the liquid sample to the device, using a plunger to urge the liquid sample through a microporous membrane, removing the membrane from the device, placing the membrane on a culture medium, incubating the culture medium, counting the number of colonies of microorganisms on the culture medium. In these embodiments, the device comprises a plunger dimensioned to provide an essentially watertight fit within a body, a microporous membrane, a removable support configured to position a microporous membrane in a flow path, and a body comprising a chamber wall and a base configured to attach to the removable support. The removable support comprises a porous support structure and a drain. In these embodiments, the body, the microporous membrane, the porous support structure, and the drain define a flow path for a liquid sample. In these embodiments, the device is configured to hold a predetermined volume of liquid sample, which is bounded at least in part by the removable support configured to position a microporous membrane in a flow path.

The words “preferred” and “preferably” refer to embodiments of the invention that may afford certain benefits, under certain circumstances. However, other embodiments may also be preferred, under the same or other circumstances. Furthermore, the recitation of one or more preferred embodiments does not imply that other embodiments are not useful, and is not intended to exclude other embodiments from the scope of the invention.

The terms “comprises” and variations thereof do not have a limiting meaning where these terms appear in the description and claims.

Unless specified or limited otherwise, the term “coupled”, “attached”, “connected” and variations thereof is used broadly and encompasses both direct and indirect couplings. Further, the term “coupled” is not restricted to physical or mechanical couplings. As used herein, the term “slideably coupled” is used to describe two or more coupled objects that, while coupled, are capable of moving relative to each other.

As used herein, “a,” “an,” “the,” “at least one,” and “one or more” are used interchangeably. Thus, for example, a device that comprises “a” valve mechanism can be interpreted to mean that the device includes “one or more” valve mechanisms.

The term “and/or” means one or all of the listed elements or a combination of any two or more of the listed elements.

Also herein, the recitations of numerical ranges by endpoints include all numbers subsumed within that range (e.g., 1 to 5 includes 1, 1.5, 2, 2.75, 3, 3.80, 4, 5, etc.).

The above summary of the present invention is not intended to describe each disclosed embodiment of the present invention. The description that follows more particularly exemplifies illustrative embodiments. In several places throughout the application, guidance is provided through lists of examples, which examples can be used in various combinations. In each instance, the recited list serves only as a representative group and should not be interpreted as an exclusive list.

### BRIEF DESCRIPTION OF THE DRAWINGS

The present invention will be further explained with reference to the drawing figures listed below, where like structure is referenced by like numerals throughout the several views.

FIGS. 1A and B show an exploded plan view of an exemplary embodiment;

FIG. 2 shows a perspective exploded view of an exemplary device with a sample intake port;

FIG. 3A shows the frontal plan view of the body of an exemplary device with a sample intake port and fill line indicia;

FIG. 3B shows the view of a longitudinal cross-section of the body of the exemplary device of FIG. 3A;

FIG. 3C shows the perspective view of the body of the exemplary device of FIG. 3A;

FIG. 3D shows a perspective view of the base of the exemplary device of FIG. 3C;

FIG. 4A shows the frontal plan view of the removable support of the exemplary device of FIG. 1;

FIG. 4B shows the longitudinal cross-section of the removable support of the exemplary device of FIG. 1;

FIG. 4C shows the upper perspective view of the removable support of the exemplary device of FIG. 1;

FIG. 4D shows the perspective view of the removable support of the exemplary device of FIG. 1;

FIG. 4E shows the longitudinal cross-section view of another exemplary embodiment with a removable support;

FIG. 4F shows the lower perspective view of the removable support of the exemplary device in FIG. 1;

FIG. 5 shows the perspective view of the plunger of the exemplary device of FIG. 1;

FIG. 6A shows the longitudinal cross-section view of an exemplary device with a valve mechanism positioned for sample intake; and

FIG. 6B shows the longitudinal cross-section view of the exemplary device of FIG. 6A with the valve mechanism positioned for sample filtration and filtrate discharge;

FIG. 7A shows an exploded plan view of an exemplary device with a microporous support structure and exit port incorporated into a plunger;

FIG. 7B shows an exploded plan view of an alternative embodiment of the device of FIG. 7A; and

FIG. 8 shows a longitudinal cross-section view of the plunger with integrated microporous support structure and exit port of FIG. 7A;

FIG. 9 shows a perspective exploded view of one embodiment of a device with a plunger that can be slideably coupled to the body.

### DETAILED DESCRIPTION

The present invention includes devices for collecting and concentrating liquid samples, such as water or other liquid beverages, and essentially homogeneous liquid suspensions of solid samples, such as food, for microbiological analysis. Because of their design, portability, and the simplicity with which they operate, the devices are particularly suitable for use in a non-laboratory environment, where the user may have limited or no access to electricity, vacuum pumps, or other accessories that are typically used to process liquid samples. The present invention also includes methods of collecting and processing liquid samples using the inventive devices.

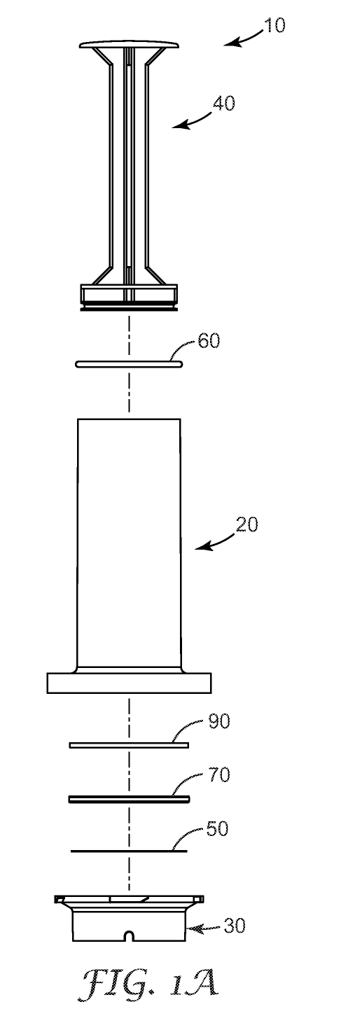

FIG. 1A and FIG. 1B show exploded views of the component parts of an exemplary device (10). The device (10) comprises a hollow, elongated body (20), which attaches to a removable support (30). A plunger (40) is shaped and proportioned to fit within and move longitudinally through the interior of the body (20). The removable support (30), onto which a microporous filtration membrane (50) can be placed, is detachably attached to the body (20). At the lower end of the plunger (40) there is a sealing ring (60). At the lower end of the body (20) there is a sealing gasket (70). The sealing ring (60) and the sealing gasket (70) keep the device (10) sealed to prevent leakage during its use and may be, where appropriate, produced from elastomeric materials, such as thermoplastic elastomers commercialized by ADVANCED ELASTOMER SYSTEMS (based in Akron, Ohio, United States) under the commercial name SANTOPRENE™; acrylonitrile and butadiene copolymers, also known as buna N and commercialized by GOODYEAR TIRE & RUBBER CO. (based in Akron, Ohio, USA) under the commercial name CHEMIGUM™; or silicon rubbers, such as rubber commercialized by DOW CORNING (based in Midland, Mich., USA). In certain embodiments, a prefilter (90) as shown in FIG. 1A, can be positioned in the device (10) in a location that is upstream in the flow path, relative to the microporous membrane (50).

The microporous membrane (50) should be porous and made, for example, out of polyamides, polytetrafluoroethylene, cellulose esters, and acetates. Appropriate membranes are manufactured by the 3M Company (based in St. Paul, Minn., USA) under the commercial name ZETAPOR, SterASSURE, BioASSURE, among others. Microporous membranes (50) are available in several standard sizes and porosities. In general, the microporous membrane will be selected to be compatible with the sample material. For example the sample should not significantly degrade the microporous membrane and the microporous membrane should not contain antimicrobial chemicals or surfactants that would significantly affect the recovery of the microorganisms to be analyzed. The porosity of the microporous membrane (50) should be selected for the liquid sample and the microorganisms to be analyzed. Preferably, the microporous membrane (50) will have a porosity of 1.0 μm or smaller. More preferably, the microporous membrane (50) will have a porosity of about 0.45 μm.

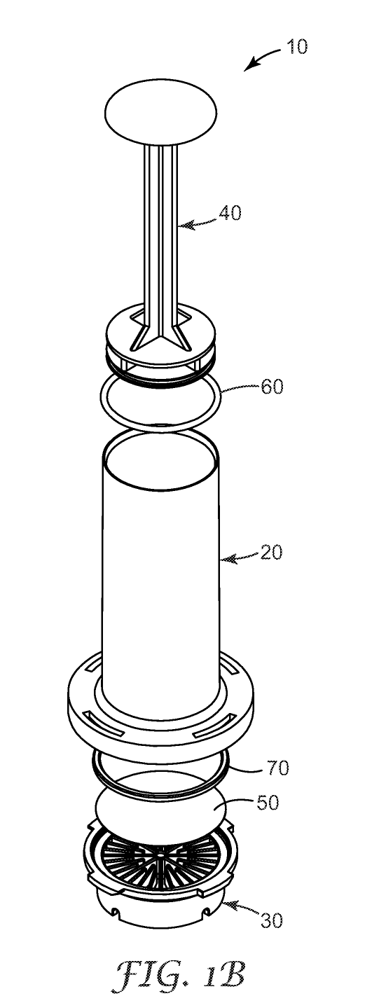

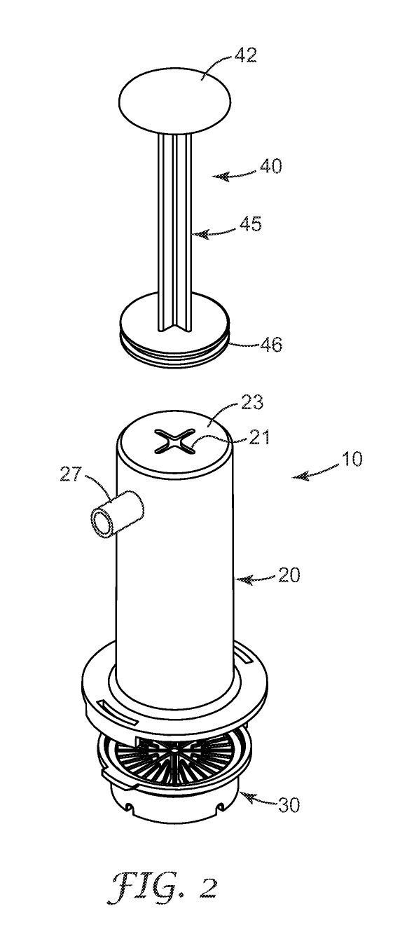

FIG. 2 illustrates an alternative unassembled embodiment of the device (10), with a body (20) further comprising a cap (23). In certain embodiments, the cap (23) is detachably attached to the body (20). The plunger (40) comprises a handle (42) and a lower base (46). In this embodiment, either the lower base (46) is removably attached to the plunger rod (45). During assembly, the lower base (46) of the plunger (40) can be detached while the plunger rod is passed through the plunger orifice (21) into the interior of the body (20). Then, the lower base (46) can be attached to the plunger rod (45) in the interior of the body (20). The microporous membrane (not shown) can be placed in the removable support (30), which is attached to the body (20) during assembly of the device (10) for use. In this embodiment, the handle (42) of the plunger (40) is located external to the body (20). In the illustrated embodiment, the plunger orifice (21) is shaped to complement the shape of the plunger rod (45). Although shown in the shape of an “X” in FIG. 2, the shape of the orifice (21) can be any shape, such as circular, rectangular, hexagonal, or the like, that complements the shape of the plunger rod (45). Alternatively, the plunger orifice comprises the opening through which the assembled plunger (40) is inserted into the body (20), as shown in FIGS. 3B-C below. Also shown in FIG. 2 are the sample intake port (27) and removable support (30), which are described in detail below.

FIGS. 3A-D show structural details of one embodiment of the body (20). The body (20) comprises chamber, defined by at least one a chamber wall (22), which is connected to a base (24). Although shown at one end of the body (20) in FIGS. 3A-D, it should be noted that the base (24), which functions as an attachment point for the removable support (not shown in FIGS. 3A-D), could be located at other positions along the body (20). In some embodiments, the chamber wall (22) is cylindrical and elongated and may be made from any suitable polymeric material, such as polypropylene, polyethylene, polyester, among others. In certain embodiments, the chamber walls (22) of the body (20), when attached to the removable support (30) that positions a microporous membrane (not shown in FIG. 3A-D) in a flow path, defines a predetermined volume of liquid sample. As shown in FIGS. 3B and 3D, projection clamps (28) and a sealing rim (29) are located within the base (24). The sealing rim (29) defines the circumference of the lower end of the chamber walls (22). In this embodiment, the sealing rim (29), in conjunction with removable support (30) and sealing gasket (70), forms a watertight seal during use.

FIGS. 3A-C depict fill line indicia (25), which indicate at least one predetermined volume of liquid in the body (20). Fill line indicia (25) may be located on the inner surface, the outer surface, or both the inner and outer surfaces of the chamber walls (22). The fill line indicia (25) can be formed, for example, as a ridge or an indentation, of the same material as the chamber walls (22). Alternatively, for example, fill line indicia (25) can be attached, printed, or etched onto the chamber walls (22). In certain embodiments, at least a portion of the body (20) is formed using transparent or translucent materials, making the meniscus of the liquid held within the body (20) visible to the operator.

FIGS. 3A-C further depict a sample intake port (27) through which liquid samples may be transferred into the body (20) of device (10). In these illustrated embodiments, the sample intake port (27) is located proximate the base (24) of the body (20). In other embodiments, such as the one shown in FIG. 2, the sample intake port (27) may be located near the end of the body (20) that is positioned further from the base (24). The sample intake port (27) shown in FIGS. 2 and 3A-C is shown as extending outward from the chamber walls (22). In some embodiments (not shown), the sample intake port (27) consists essentially of an opening in the chamber walls (22). The sample intake port (27) may be formed from the same material and as an integral part of the chamber walls (22). Alternatively, the sample intake port (27) may be formed independently and subsequently attached to a suitably-sized and positioned opening in a chamber wall (22).

In some embodiments, the sample intake port (27) comprises a hose, pipe, tubing, or the like, to provide a passageway through which a liquid sample can be transported. In other embodiments, the sample intake port (27) further comprises a valve such as, for example, a ball valve, a check-valve, or a spigot, to regulate the velocity, volume, and/or direction of liquid flow, or even to stop liquid flow completely. In certain embodiments, the sample intake port (27) further comprises a prefilter, to reduce the number of larger, particulate materials in the sample material. Suitable prefilters include, for example, microporous membranes and/or cartridge filters with the appropriate porosity to permit passage therethrough of the target microorganisms to be analyzed.

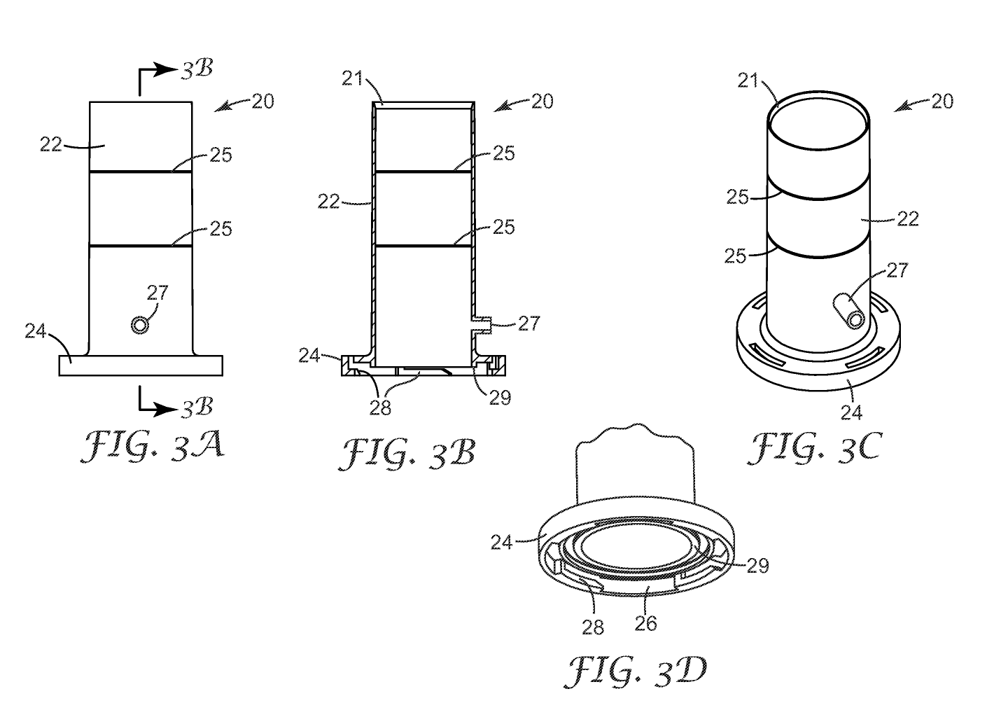

FIGS. 3B-C show at one end of the body (20), there may be a plunger orifice (21), into which the assembled plunger (not shown) can be inserted. At the other end of the body (20), there is a base (24). The exemplary base (24) has projection clamps (28) and projection slots (26), as shown in FIG. 3D. The projection clamps (28) enable the body (20) to be affixed to the removable support (30). When assembling the exemplary device (10), the projections (32) of the removable support (30) shown in FIGS. 4C-D are aligned with and inserted into the projection slots (26), and either the base (24) or the removable support (30) is rotated to move the projections (32) into the narrow opening situated above the projection clamps (28) shown in FIG. 3C.

FIGS. 4A-D show details of an exemplary removable support (30). The removable support (30) is attached to the body (20) of the device (10) in a manner such that it can be detached from the same. The structures or methods used for the removable attachment and detachment are generally mechanical and may have diverse configurations (not shown) such as, for example, through pins and balls, mechanical fixing systems of the hook and loop type, bolt and screw systems. Other suitable structures for enabling the body (20) and the removable support (30) to be removably affixed to each other are known in the art, and may be used with the present invention. In some embodiments, the removable support (30) possesses projections (32) which may be fitted into projection clamps (28) on the base (24) of the body (20). The removable support (30) also presents a filtrate drain (31) for discharge of the filtered matter, after is has passed through the microporous membrane (50). In some embodiments as shown in FIGS. 4A, 4B, and 4D, the removable support comprises a drain housing (33) and optional drain holes (34) to facilitate the passage of liquid and/or air from the drain housing (33). The drain hole (34) can be configured to have a variety of shapes, numbers, and sizes. The drain holes (34) provide an egress for filtrate when a removable support (30) similar to that shown in FIG. 4A is placed on an essentially smooth, flat surface during the use of device (10).

In alternative embodiments, one of which is shown in FIG. 4E, the removable support (30) comprises an exit port (35) located on the radial perimeter of the drain housing (33), creating a liquid flow path that may be essentially perpendicular to the flow path within the chamber walls (22). In these embodiments, the exit port (35) may comprise an opening in the radial perimeter of the removable support (30) or alternatively, the exit port (35) may further comprise an extension, as shown in FIG. 4E. The exit port (35) optionally comprises a hose, a pipe, tubing, or the like, to provide a path through which a liquid sample can be transported. In some embodiments, the exit port (35) further comprises a valve such as, for example, a ball valve, a check-valve, or a spigot, to regulate the velocity, volume, and/or direction of liquid flow, or to stop liquid flow completely.

In some embodiments, the exit port (35) further comprises an optional barrier seal. Exemplary barrier seals include a water-resistant film or sheet adhesively adhered or removably bonded to the filtrate drain (31) or, if present on the removable support (30), the exit port (35). Alternatively, the barrier seal may be a plug, which is suitably proportioned to be removably secured into the filtrate drain (31) or the exit port (35). The plug can be made from a number of suitable materials, such as plastic or rubber. The barrier seal is removed prior to filtering the liquid sample in the device (10). The barrier seal is used to prevent the liquid sample from prematurely passing through the microporous membrane (50) and out of the filtrate drain (31) or the exit port (35) during handling or transport. Additionally, the barrier seal helps prevent materials from entering through exit port (35) and/or the filtrate drain (31) and causing chemical or microbial contamination of the microporous membrane (50).

Prior to using the device (10) to concentrate a liquid sample, a microporous membrane (50) is placed on or, preferably, inside the removable support (30) (as shown in FIG. 1A-B). The microporous membrane (50) is preferably supported essentially perpendicular to the liquid flow by a shelf (36) and crossbars (38) formed on the internal wall of the removable support (30), although any suitable arrangement by which liquid can be directed through the membrane may be used. The shelf (36) and crossbars (38) essentially form a porous support structure to position a microporous membrane in a liquid flow path. The thickness and diameter of the microporous membrane (50) should be compatible with the diameter of the shelf (36) of the removable support (30) and the diameter of the sealing gasket (70). In the assembled device (10), the sealing gasket (70) is preferably positioned on top of the outer rim of the microporous membrane (50), forming an essentially watertight seal between the microporous membrane (50) and the sealing rim (29).

In the illustrated embodiment, the removable support (30) includes crossbars (38) which project longitudinally downward from the upper end of the removable support (30) to a floor (37) and extend radially inward from the shelf (36). In this embodiment, the floor (37) has a central opening, the filtrate drain (31), which helps to direct the liquid flow out of the removable support (30), as shown in FIG. 4C. As shown in FIGS. 4C-D, the shelf (36) has the form of a solid ring inside the removable support (30) and, in conjunction with the sealing gasket (70) and the sealing rim (29) of the body (20), the shelf (36) aids in forming a watertight seal.

The crossbars (38) provide a porous support structure for the microporous membrane (50) during the use of the device (10). Although shown as crossbars (38) in FIG. 1A-B, the porous support structure may have various other configurations. An alternative design (not shown) may include a single support member consisting of an essentially solid, preferably planar surface, with a plurality of orifices, which provide passages, for liquid flow out of the removable support, spaced across the surface. The porous support structure provides sufficient support for the microporous membrane (50), so that the membrane does not break or pass through the orifices in the porous support structure during the process in which hydrostatic pressure is applied to the microporous membrane (50).

FIG. 4F shows a lower perspective view of the removable support (30). The projections (32) can be used to attach the removable support (30) to the body (20). During use, the liquid filtrate passes through the microporous membrane (50) into the spaces between the crossbars (38), until the filtrate is directed by the floor (37) to the drain opening (31). The drain opening (31), drain housing (33), and drain holes (34) provide liquid flow paths for the sample filtrate to exit the removable support (30).

The removable support (30) and its components may be produced, in an appropriate form, from polymeric materials. Nonlimiting examples of such materials include polypropylene, polyethylene, polyester and polycarbonate.

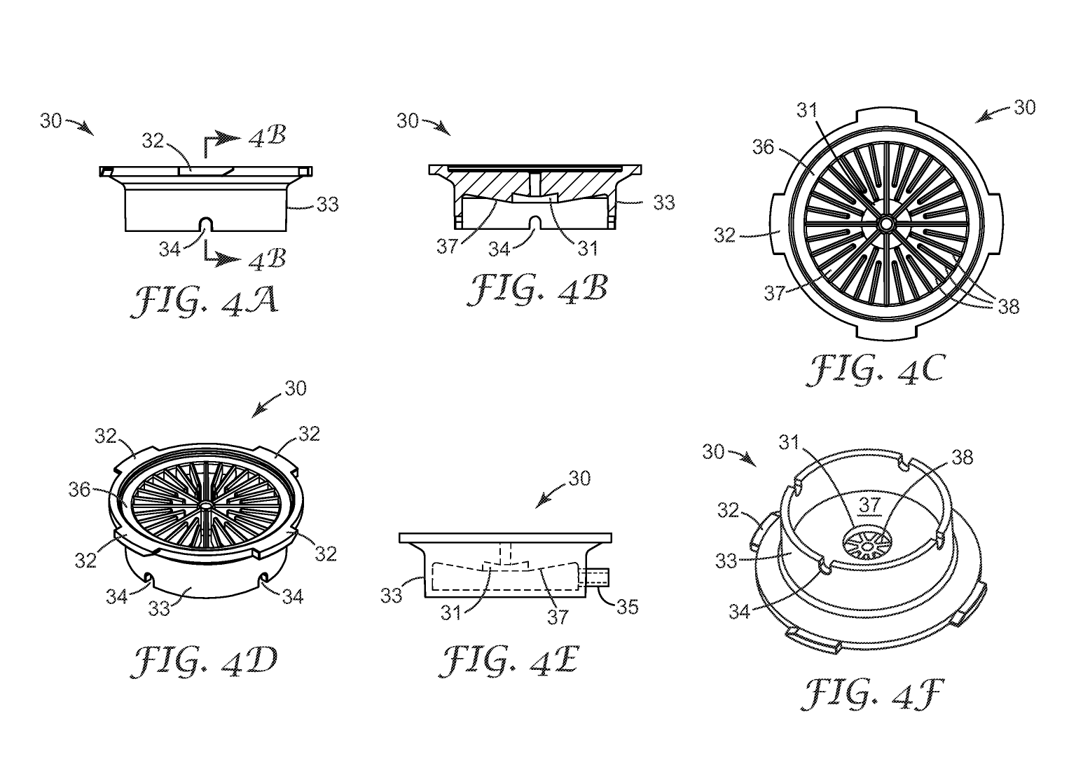

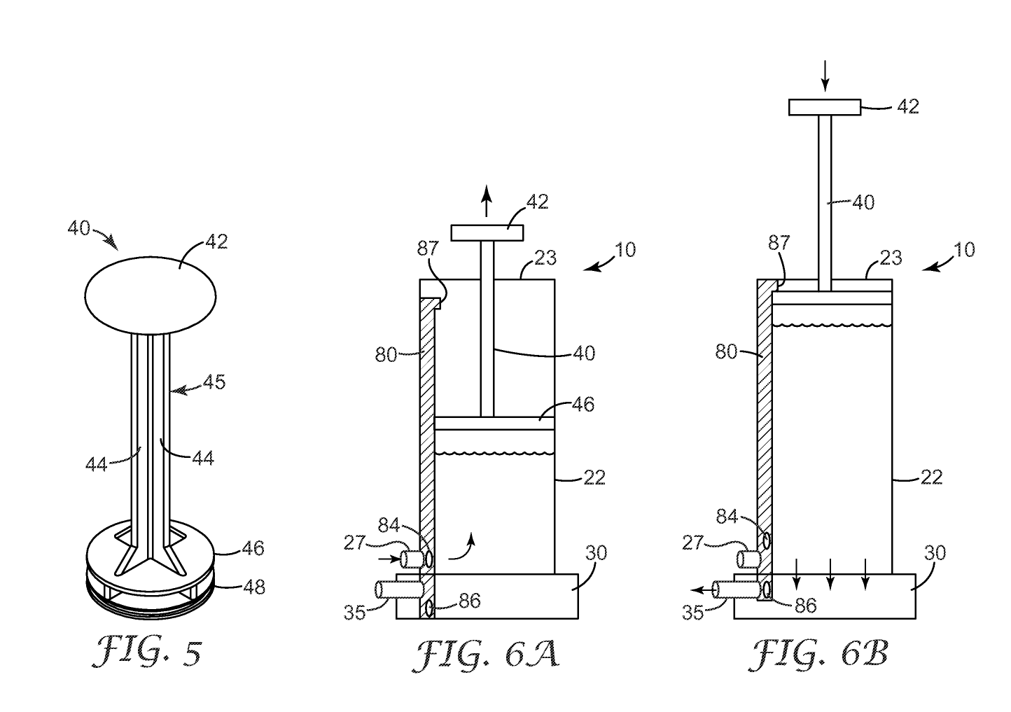

FIG. 5 shows an exemplary embodiment of the plunger (40), which comprises a structure which is adapted to fit within the cavity of the body (20). When plunger (40) is moved downward as shown in FIG. 6B, the plunger (40) exerts pressure on the sample collected and causes the sample to pass through the microporous membrane (50) positioned on the inside of the removable support (30) of the device (10).

The plunger (40) comprises a plunger rod (45), a handle (42) and a lower base (46), respectively. The lower base (46) preferably has a slot (48) where the sealing ring (60) is placed. The sealing ring (60) is formed from elastomeric material and is adapted to fit into and be retained within the slot (48). When the sealing ring (60) is positioned in the slot (48), the lower base (46) of the plunger (40) fits within the cavity of the body (20) to provide an essentially watertight seal as the plunger (40) moves longitudinally through the interior of the body (20). The handle (42) of the plunger (40) functions as a surface on which force is exerted to propel the plunger (40) longitudinally in either direction through the body (20). The form of the plunger (40) may vary according to the need or desire of the consumer, and, may for example be formed of a solid body or have a substantially open construction, such as the plunger rod (45) comprised of longitudinal fins (44), as shown in FIGS. 1, 2 and 5. One advantage of a substantially open structure is the relatively low cost of fabrication of the part, since less material is used in its manufacture. The plunger (40) may be produced using polymeric materials with suitable properties, such as strength, weight, and adaptability to manufacturing processes. Non-limiting examples of such materials with suitable properties include such as polypropylene, polyethylene, and polyester.

The plunger (40) may comprise optional fill line indicia (not shown). The plunger fill line indicia may be formed of the same material as the plunger rod (45) in the shape of, for example, ridges or indentations. Alternatively, for example, plunger fill line indicia can be attached, printed, or etched onto the plunger rod (45). When the sample is drawn into the device (10) using the plunger, as described below, the alignment of a plunger fill line indicium with, for example, the cap (23) is an indication that a known volume of sample liquid is present in the body (20).

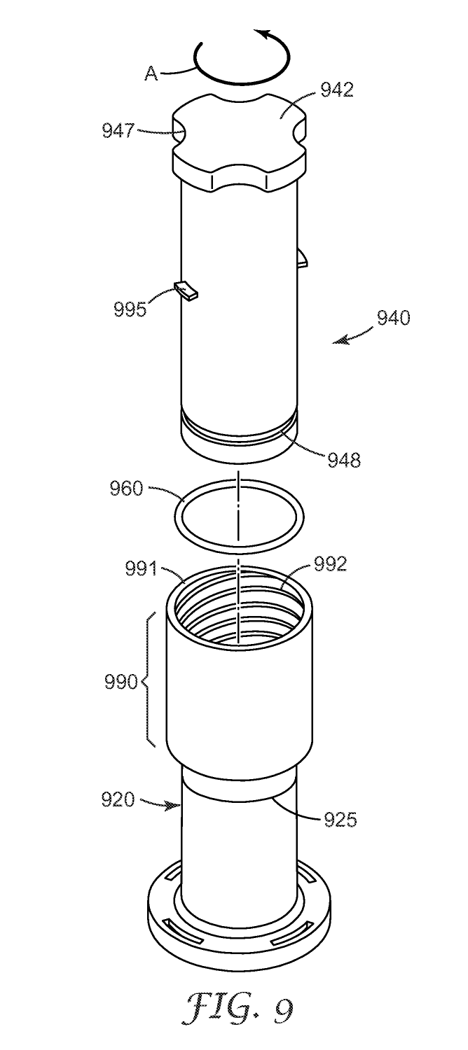

In certain embodiments, the device can be adapted to facilitate the process of filtration. In these embodiments, the plunger and the body can be slideably coupled, comprising structural elements that can be used to urge the liquid sample through a microporous membrane. The embodiments can provide a mechanical advantage for the filtration step, making it easier for the operator to apply pressure to the plunger during filtration. This can be particularly advantageous when filtering viscous samples or samples with relatively large numbers of particulates. FIG. 9 shows a body 920 which includes a coupling region 990. In this embodiment, the coupling region 990 comprises a continuous helical groove 992, which spans the length of the coupling region 990. The length of the coupling region 990 can be selected such that it corresponds to a predetermined volume of sample. FIG. 9 also shows a plunger 940 that has an outer diameter proportioned to fit inside the body 920. The plunger comprises a slot 948, into which a sealing ring 960 is positioned. The sealing ring 960 can be made of various materials, such as butyl or silicone rubber, which permit the movement of the plunger 940 through the body 920 while maintaining a substantially liquid-resistant seal between the inner surface of the body 920 and the outer diameter of the plunger 940. The plunger 940 also comprises high-relief tracks 995 and a handle 942 with concavities 947. The tracks 995 extend from the handle 940 and are proportioned to fit in the groove 992 and, thus, the plunger 940 can be slideably coupled to the coupling region 990 of the body 920. The concavities 947 provide a convenient surface for the user to grasp and rotate the handle 942 during use. Also shown in FIG. 9 is a fill line indicium 925. When fully assembled for use (not shown), the body 920 is coupled to a removable support containing a microporous membrane, through which the sample is passed.

During use, the operator can add a liquid sample to the body 920. Optionally, the operator can add a predetermined volume of sample by filling the body 920 to the fill line indicium 925. The plunger 940 is inserted into the body 920 until the tracks 995 contact the upper edge 991 of the coupling region 990. The handle 942 is then rotated in the direction of arrow A to slideably move the tracks 995 into the groove 992. The operator can continue to rotate the handle 942 until the entire sample (e.g., a predetermined volume) has passed through the microporous membrane (not shown).

A skilled person will recognize that various alternative structures can be used to accomplish the same function as the structures shown in FIG. 9. For example, a person could use only one track 995 or numerous tracks 995 that are proportioned and aligned such that they allow the plunger 940 to be slideably coupled to the body 920 in the coupling region 990. Additionally, the tracks 995 could be replaced with a helical ridge (not shown), such that the ridge could slideably couple to the groove 992 in a configuration similar to a nut and a bolt.

Certain embodiments of the device (10) comprise a valve structure to regulate the flow of liquid into and out of the device (10). One example of a valve structure to regulate fluid flow within the device (10) is shown in FIGS. 6A-B. In FIG. 6A, the device (10) is shown with the configuration of the valve regulator (80) and the path of fluid flow during the filling process. Prior to the filling process, in which the liquid sample is drawn into the chamber (22), a first valve opening (84) is aligned with the sample intake port (27), providing a fluid path into the body (20). Additionally, the plunger (40) and the valve actuator (87) are in their respective first positions. During the filling process, force is applied to the handle (42) to pull the plunger (40) in a direction away from the removable support (30) causing the liquid sample to be drawn into the body (20) through sample intake port (27). During this procedure, the exit port (35) is preferably blocked by the valve regulator (80). When the lower base (46) of the plunger (40) contacts the valve actuator (87), the valve actuator (87) is entrained with the movement of the plunger (40). The plunger (40) continues to move until the valve actuator (87) reaches a second position, preferably after filling the chamber with a predetermined volume of liquid sample, when it comes in contact with the cap (23) of the body (20). In this embodiment, the predetermined volume of the body (20) is essentially defined by the first and second position of the plunger (40).

After the filling process is completed, the device can be used to perform the emptying process. Referring to FIG. 6B, when the plunger (40) is in the second position, the valve regulator (80) is positioned such that the second valve opening (86) is substantially in alignment with the exit port (35) of the removable support (30), creating a fluid flow path to allow liquid to pass out of the device (10) via the exit port (35). During this process, pressure is applied to the handle (42), causing the plunger (40) to move toward the removable support (30). This movement causes the liquid sample to preferably pass through the microporous membrane (not shown in FIG. 6B), and out of the removable support (30) via the exit port (35). The passage of the liquid sample through the microporous membrane facilitates the collection and concentration of microorganisms from the liquid sample onto the microporous membrane.

In the illustrated embodiment, the chamber walls (22), the valve regulator (80), the lower base (46) and the sealing ring (not shown in FIGS. 6A-B) are formed such that the plunger (40) can move within the body (20) during the filling process, while maintaining an essentially watertight seal, without causing significant movement of the valve regulator (80) until the valve actuator (87) is contacted by the lower base (46). Similarly, during the emptying process, the plunger (40) can move within the body (20), maintaining an essentially watertight seal, without causing significant movement of the valve regulator (80).

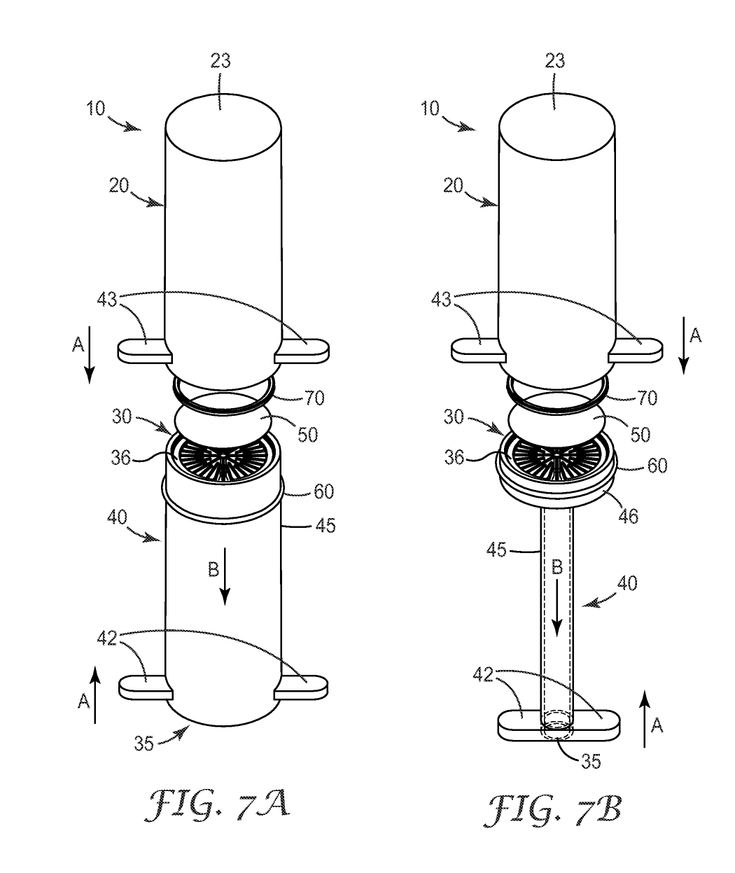

In the illustrated embodiments shown in FIGS. 7A-B, the removable support (30) can be incorporated into the plunger (40). In these embodiments, the body (20) is preferably sealed at one end with a cap (23). The body (20) may also comprise handles (43) to grasp while inserting and depressing the plunger (40). At one end of the plunger (40) are handles (42) and at the other end of the plunger (40) is a removable support (30) comprising a porous support structure.

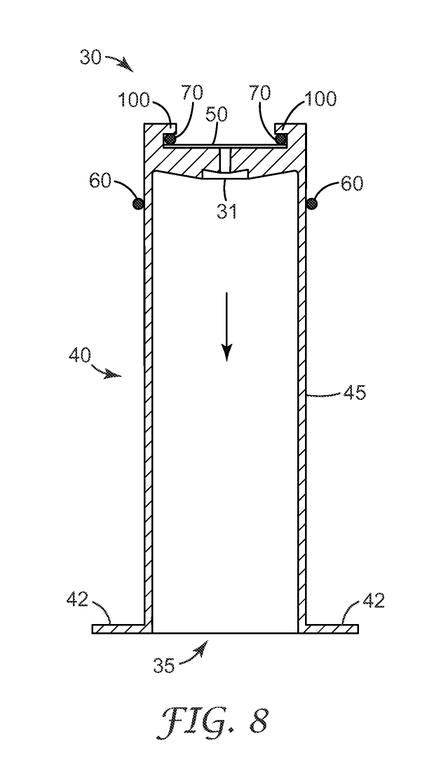

FIG. 7A illustrates a removable support (30) integrated into a plunger (40), whereby the plunger rod (45) preferably forms a hollow passageway for the liquid sample filtrate. In this embodiment, the outer diameter of the plunger rod (45) is slightly smaller than the inner diameter of the body (20). A sealing ring (60) surrounding the removable support (30) provides an essentially watertight seal when the plunger (40) is inserted into the body (20). An advantage of this embodiment is that the plunger (40) provides a large passageway from the drain (31) (shown in FIG. 8) to sample exit port (35) through which the liquid sample filtrate can exit the device (10). The large passageway to sample exit port (35) may provide for high volume throughput and low back-pressure for the liquid passing out of the device (10). Additionally, plunger rods (45) that are longer than the body (20) may comprise an internal volume that is large enough to hold the entire predetermined volume of the sample filtrate. When filtration is complete in those embodiments, the device may be moved to a convenient location to dispose of the filtrate.

FIG. 7B illustrates a removable support (30) integrated into a plunger (40), whereby the plunger rod (45) preferably forms a hollow passageway from the drain (31) (shown in FIG. 8) to sample exit port (35) for the liquid sample filtrate. In this embodiment, the outer diameter of the plunger handle (40) is significantly smaller than the inner diameter of the body (20). A sealing ring (60) surrounding the removable support (30) provides an essentially watertight seal when the plunger (40) is inserted into the body (20). An advantage of this embodiment is that it can be constructed using less material than the device (10) in FIG. 7A and that the sample exit port (35) can be attached to appropriately-sized tubing to carry the liquid filtrate to a drain or an appropriate receptacle.

In an alternate embodiment (not shown), the drain could be the sample exit port. In a further alternate embodiment (not shown), the drain may be connected to the sample exit port though a hollow passageway (for example, hollow tubing) separate from the plunger rod. In this embodiment, the plunger rod may or may not be a hollow passageway, as the liquid sample filtrate exits through the drain on the removable support.

Prior to use, a microporous membrane (50) is placed on the removable support (30) and is held in place with a sealing gasket (70) or other suitable secural means. FIG. 8 shows a plunger (40) with plunger handles (42) and a modified removable support (30) adapted to secure the microporous membrane (50). The microporous membrane (50) is placed onto the surface of the removable support (30) and the sealing gasket (70) is positioned under a ledge (100) that overhangs the shelf (36) (shown in FIG. 7A). Thus, the sealing gasket (70) and the microporous membrane (50) are held in place, with the microporous membrane (50) positioned in a flow path such that a liquid sample may pass through the microporous membrane (50), the drain (31), the plunger rod (45), and out the sample exit port (35). Also shown in FIG. 8 is the sealing ring (60), which forms an essentially watertight seal when the plunger (40) is inserted into the body (20).

As described above, elements of the present invention may form at least a part of the boundary of a predetermined volume of a liquid sample. Various combinations of the elements form the entire boundary of a given predetermined volume. These elements can comprise the chamber walls (22), the lower base (46) of the plunger (40), the removable support (30), the plunger opening (21), fill line indicia (25), and a valve (80). When present in the device (10), a microporous membrane (50) may act in concert with the removable support (30) to form at least a part of the boundary of a predetermined volume of liquid sample.

### Sample Collection and Concentration

To illustrate the present invention, the individual steps in the methods presented below are shown to follow a particular sequence. It should be recognized that, depending upon the particular device, certain individual steps may be conducted in a different order, as will be evident in the following description.

Certain embodiments of the invention begin with the placement of the sample to be analyzed into the cavity of the body (20). The sample may be drawn into the chamber of the body in the manner described above, or the plunger (40) may be removed from the device (10) and the sample may be placed into the body (20). In certain preferred embodiments, the barrier seal, if present, is left in place until the sample has been transferred into the body (20) and the operator is ready to commence the filtration process. The sample may be placed directly into the device (10) without the use of an intermediate sample collector. This can be done, for example, by dipping the device (10), with the plunger (40) removed, directly into a sample source, such as a lake, a pond, a stream, a river, an ocean, a tank, a vat, a pot, a carboy, a reservoir, a cistern, a bucket, or any other body of liquid. When obtaining the sample by dipping, the barrier seal helps prevent exposure of the microporous membrane to sample material that is not contained within the predetermined volume in the device (10). Alternatively, the sample can be transferred directly into the device (10) by placing the device under a spigot, a hose, a pipe, or the like and flowing the sample, using gravity or pressure generated by other means such as a pump, into the body (20).

Alternatively, the sample can be transferred into the device using an intermediate sample collector. An intermediate sample collector is a device that is used to collect, and temporarily hold, the sample prior to depositing it into the body (20) of the device (10). Nonlimiting examples of intermediate sample collectors include test tubes, flasks, beakers, buckets, bottles, syringes, and pipettes. In these embodiments, the sample is placed in or collected directly into the intermediate sample collector and, thereafter, poured, pumped, or drained into the device (10).

After filling the body (20) with the liquid sample, excess liquid can be poured out until the sample is level with one of the fill line indicia (25). In certain embodiments, the entire cavity of the body (20) defines a predetermined volume and there is no need to pour out excess liquid.

After the sample is collected into the body (20), the barrier seal, if present, can be opened and/or removed. The plunger (40) is then placed into the upper end of the body (20) of the device (10) and pressure is applied to the handle (42) of the plunger (40) to propel the plunger (40) longitudinally through the inside of the body (20). The pressure may be applied to the handle (42) either manually or through the use of an appropriate machine designed to impart an appropriate amount of pressure to propel the sample through the microporous membrane (50) and to cause the plunger (40) to traverse the length of the body (20). A skilled person will recognize that the amount of pressure applied to the handle should be enough to cause the liquid sample to move through the microporous membrane (50) at an acceptable rate but not enough pressure to cause the microporous membrane (50) to rupture or to cause significant leakage from the sealing ring (60) or the sealing gasket (70). The liquid sample, under pressure, passes through the microporous membrane (50), and is thus filtered. The microorganisms contained in the sample are retained in the microporous membrane (50) in concentrated form. The removable support (30) may then be detached from the body (20) and the microporous membrane (50) removed for microbiological analyses. The microporous membrane (50) can be removed from the device (10) immediately, or it can be stored temporarily in the device (10) until microbiological analyses are conducted at a later time.

In the embodiments where the device (10) comprises a sample intake port (27), the sample intake port (27) can be placed in fluid communication with the sample by, for example, immersing the sample intake port into the body of liquid from which the sample will be taken. Alternatively, the sample intake port (27) can be connected to a passageway, such as tubing, a pipe, a valve, or the like, through which a sample can be drawn or pumped into the body (20). In certain embodiments where the sample intake port is positioned proximate the base (24) of the body (20), as shown in FIG. 3C, the plunger (40) is fully inserted in the body (20) prior to collecting the sample. After the sample intake port (27) is placed in fluid communication with the source of the liquid sample, the plunger (40) is withdrawn from the body (20) until the liquid sample fills the body (20) to the appropriate fill line indicium (25) on the chamber wall (22) or the plunger (40). A valve is then actuated, either manually or automatically, to prevent fluid flow through the sample intake port (27). Force is applied to the plunger (40) to cause the liquid sample to flow through the microporous membrane (50). After the liquid sample has been filtered, the removable support (30) is detached from the chamber base (24) and the microporous membrane (50) is removed for microbiological analyses.

In the embodiments where the device (10) comprises a valve structure to regulate the flow of liquid sample into and out of the device (10), the process preferably begins with the plunger (40) fully inserted into the body (20). As the plunger (40) is moved through the body (20) in a direction away from the removable support (30), the sample is drawn into the body (20) until the sample volume reaches the fill line indicium. The valve is closed, either manually or automatically, and force is applied to the plunger (40) to urge it toward the removable support (30). After the entire sample has passed through the microporous membrane (50), the removable support (30) is detached from the body (20) and the microporous membrane (50) is removed for microbiological analyses.

### Samples

In some embodiments, samples to be collected and analyzed are samples from a body of water. Nonlimiting examples of such bodies of water include surface water, water for human or animal consumption, and water used for industrial processes. Surface water includes an ocean, a lake, a river, a canal, a pond, a reservoir, a stream, and the like. Process water includes water that is used in municipal or industrial purposes, such as cleaning, washing, rinsing, cooling towers, water treatment holding tanks, and the like. Exemplary cleaning processes include food processing processes, such as, washing, rinsing, and disinfecting meat or produce for human or animal consumption.

In other embodiments, the devices and methods of this invention are used to collect and analyze any liquid sample that is amenable to filtration, such as, for example, solutions, mixtures, homogenates, or liquid suspensions of foodstuffs, beverages and pharmaceutical products, provided the liquid sample does not cause undue clogging or degradation of the microporous membrane (50). In certain embodiments, the liquid sample comprises one or more dissolved solute, such as sugars, salts, or proteins. In other embodiments, the liquid sample comprises one or more solvent, such as an alcohol, or a surfactant. Samples with solvents or surfactants can be used in accordance with the present invention, provided the solvents or surfactants do not significantly impair the filtration properties of the microporous membrane (50) or degrade or decompose the materials from which the device (10) is constructed. Preferably, the sample is substantially free of particulate materials that could clog the microporous membrane (50).

Another feature of the invention is the variety of predetermined sample volumes that can be tested. Preferably, the volume of the sample is large enough to be distributed over the entire surface of the microporous membrane (50). The body (20) of the device (10) can be designed with various capacities. Preferably, the body (20) is designed to hold approximately 50 to 250 milliliters. More preferably, the body (20) is designed to hold approximately 100 milliliters. When it is desirable to test liquid sample volumes that exceed the capacity of the body (20) of the device (10), the method of use provides that more than one aliquot of the sample can be loaded sequentially into the body (20) and filtered through the microporous membrane (50), provided that the user takes care to minimize or avoid the introduction of nonindigenous microorganisms when introducing the additional aliquots of sample liquid. When it is desirable to test very small liquid sample volumes, the method of use provides that any small liquid sample can be added to a suitable diluent. Preferably, the diluent is sterile prior to the addition of the sample liquid. Nonlimiting examples of such diluents include distilled water, deionized water, reverse-osmosis (RO) water, physiological saline, phosphate-buffered saline, Standard Methods Buffer, Butterfield's Buffer, and the like.

Individual liquid samples may contain almost any number and kind of microorganism. The number of microorganisms in a liquid sample may range from zero organisms per milliliter, in a sample that has been subjected to sterilizing conditions, up to approximately 10⁹ or more organisms per milliliter in a heavily-contaminated sample. The devices and methods of the present invention provide for the concentration and analysis of liquid samples containing a wide variety of bacterial concentrations. One of the primary determinants of sensitivity of the methods is the volume of liquid sample that is passed through the microporous membrane (50). As mentioned above, certain embodiments of the present invention provide for the filtration of multiple volumes of liquid sample. This aspect permits the user to obtain sensitive detection of a single bacterium in, for example, 100 milliliters, 250 milliliters, 500 milliliters, 1 liter, or more of sample liquid. Liquid samples containing relatively high concentrations of bacteria can be diluted appropriately, so that the number of bacteria in the sample does not significantly interfere with the filtration process.

In certain embodiments, the fluid samples comprise a food or beverage. Methods for the preparation of food samples for microbiological analyses are well known. Some of the sample preparation methods for food samples involve suspending a known quantity of food material (25 grams, for example) in a relatively large volume of diluent (225 milliliters, for example). The sample is subjected to a strenuous mixing process, such as blending or stomaching, to create a relatively homogeneous liquid suspension. Devices of the present invention provide a way to concentrate and analyze food or beverage liquid samples; provided that the amount of suspended particulates or the viscosity of the sample is not at a level that is high enough to significantly interfere with the filtration process.

### Microbiological Analyses

In one embodiment of the invention, the microbiological analyses can be conducted immediately after the sample has been collected and concentrated. After the sample has been collected and concentrated in the device (10), the microporous membrane (50) is removed for microbiological analysis. Preferably, the microporous membrane is placed into a device with semisolid microbiological culture medium. Nonlimiting examples of such devices include Petri dishes containing various agar media, Petri dishes containing Easygel® media (Micrology Laboratories, Goshen Ind.), and several types of dry, rehydratable culture media, such as 3M™ PETRIFILM™ Aerobic Count Plates (3M Company, St. Paul, Minn.), 3M PETRIFILM Coliform Count Plates, 3M PETRIFILM Coliform/E. coli Count Plates, Compactdry Total Count Plates (Nissui Pharmaceutical Company, Ltd., Tokyo, JP), Sanita-kun® Coliforms Plate (Chisso Corporation, Tokyo, JP), and the Sanita-kun Total Aerobic Count Plate. Preferably, the dry, rehydratable culture media are rehydrated prior to inserting the microporous membrane (50) from the device (10). An advantage of the present invention is that the portable, easy to use device (10) can be used with easy to use rehydratable culture media to perform the microbiological analyses in a field location with minimal laboratory equipment, such as a small area or glove box for aseptic transfer of the microporous membrane (50) onto the culture media. Additionally, a small incubator could provide temperature control for the incubation of the culture media in a field location.

Subsequent to the placement of the microporous membrane (50) onto the culture medium, the culture medium is incubated at an appropriate temperature for an appropriate time for the growth of colonies of microorganisms, as known by a person skilled in the art, and in accordance with the standard methods. The present device (10) can be used to concentrate microorganisms that are typically found in water or food samples. Microorganisms that are of particular interest in water samples include, for example, coliforms, fecal coliforms, Escherichia coli, and certain species of the genera Pseudomonas, Aeromonas, Enterococcus, Legionella, and Mycobacterium, among others. Microorganisms that are of particular interest in food samples include, for example, aerobic bacteria, coliforms, yeast, mold, lactic acid bacteria, members of the large bacterial family Enterobacteriaceae, Escherichia coli, enteropathogenic E. coli, enterotoxigenic E. coli, E. coli O157H7, and certain species of the genera Salmonella, Shigella, Vibrio, Listeria, Staphylococcus, Pseudomonas, Clostridium, Streptococcus, Yersinia, Bacillus, and Campylobacter, among others.

Tests for coliforms, for example, typically require incubation at a temperature of approximately 35° C. for 24 to 48 hours. Tests for fecal coliforms are incubated at a temperature of approximately 45° C. After the period of incubation, the microporous membranes (50) are examined for the presence of bacterial colonies and the number and type of each colony is recorded. Certain microbiological media, such as Violet Red Bile (VRB) agar contain indicators that distinguish certain bacteria, such as, lactose-fermenting bacteria, from others.

Typically, the colonies are counted manually. When available, devices such as a magnifying lens and/or a dark field magnifying device, such as a Quebec Colony Counter, can be used to assist in counting the colonies. Alternatively the plates can be counted using an automated plate counter such as, for example, ProtoCOL SR or HR colony counting systems from Synbiosis (Fredrick, Md.) or a Petrifilm Plate Reader from 3M Company (St. Paul, Minn.), provided the microporous membrane (50) and the growth medium used in the procedure are compatible with the automated colony counting system.

In other embodiments of the present invention, the sample can be collected and concentrated in the device (10) and the device (10) is transferred to a laboratory, where the microporous membrane (50) is removed for subsequent microbiological analyses.

In yet another embodiment of the present invention, the sample is collected and concentrated in the device (10) and the microporous membrane (50) is removed and placed into a sterile container for transport to a laboratory for subsequent testing. Preferably, the container is designed to keep the microporous membrane (50) moist during transport, to avoid loss of viability of the microorganisms. Optionally, a preservative can be added to the container to maintain the viability of the microorganisms during transport.

### Kits

Kits comprised of the device (10) for sampling, processing, and/or the microbiological evaluation of liquid samples are also contemplated. The kits may provide the device (10) and any one of a number of accessories that are useful in the methods of sample collection, concentration, and/or microbiological analyses. Such accessories may include, for example, gloves, labels, a bag, an intermediate sample collector, a disinfectant, a forceps, a culture medium, a microporous membrane, a prefilter, a culture medium carrier, an incubator, and a reagent. The intermediate sample collector may be comprised of, for example, a test tube, a flask, a beaker, a bucket, a bottle, a syringe, or a pipette. The kits may be pre-sterilized by the appropriate methods known in the technical field, such as irradiation, ethylene oxide and heat.

### EMBODIMENTS

1. A device for collecting and concentrating a sample for microbiological analysis, the device comprising:

a plunger dimensioned to provide an essentially watertight fit within a body;

a removable support configured to position a microporous membrane in a flow path, the support comprising
a porous support structure; and
a drain; and

a body comprising
a chamber wall; and
a base configured to attach to the removable support;

wherein the body, the porous support structure surface, and the drain define a flow path for a liquid sample;

wherein the device is configured to hold a predetermined volume of liquid sample; and

wherein the predetermined volume is bounded at least in part by the removable support configured to position a microporous membrane in a flow path.

2. A device for collecting and concentrating a sample for microbiological analysis, the device comprising:

a plunger dimensioned to provide an essentially watertight fit within a body;

a removable support configured to position a microporous membrane in a flow path, the support comprising
a porous support structure; and
a drain; and

a body comprising
a chamber wall;
a base configured to attach to the removable support;
a plunger orifice; and
a sample intake port;

wherein the sample intake port, the body, the porous support structure surface, and the drain define a flow path for a liquid sample; and

wherein the device is configured to hold a predetermined volume of liquid sample.

3. A device according to embodiment 1 or 2, wherein the removable support further comprising an exit port.

4. A device according to embodiment 1 or 2, wherein the drain further comprises a barrier seal.

5. A device according to embodiment 2, wherein the predetermined volume is bounded at least in part by the removable support configured to position a microporous membrane in a flow path.

6. A device according to embodiment 1 or 2, wherein the predetermined volume is further bounded by a chamber wall.

7. A device according to embodiment 1 or 2, wherein the predetermined volume is defined by a fill line indicium.

8. A device according to embodiment 2, wherein the sample intake port is positioned on a region of the body proximate the removable support.

9. A device according to embodiment 2, wherein the sample intake port is positioned on a region of the body apart from the removable support.

10. A device according to any of embodiments 2 through 9 wherein the sample intake port further comprises a valve.

11. A device according to embodiment 10, wherein the valve is actuated by the plunger.

12. A device according to any one of the previous embodiments, the device further comprising a microporous membrane positioned in a liquid flow path.

13. A device according to any one of the previous embodiments, the device further comprising a prefilter.

14. A device according to any one of the previous embodiments wherein the body comprises at least one fill line indicium.

15. A device according to any of embodiments 2-14, wherein the plunger comprises at least one fill line indicium.

16. A device according to embodiment 14 or embodiment 15, wherein the at least one fill line indicium defines a predetermined volume in the device.

17. A device according to any one of the previous embodiments wherein the exit port comprises a barrier seal.

18. A device according to any one of the previous embodiments wherein the porous support structure comprises a plurality of crossbars.

19. A device according to any of the previous embodiments wherein the plurality of crossbars is arranged radially.

20. A device according to any one of the previous embodiments wherein the body further comprises a gasket seal.

21. A device of any one of the previous embodiments wherein the device is pre-sterilized.

22. A device according to any one of the previous embodiments, wherein the plunger is slideably coupled to the body.

23. A process for collecting and concentrating a sample for microbiological analysis, the process comprising:

providing a liquid sample to be analyzed;

providing the device of any one of embodiments 12 through 22;

transferring a predetermined volume of the liquid sample to the body of the device; and

applying force to the plunger to urge the liquid sample through the microporous membrane.

24. A process for enumerating microorganisms in a sample, the process comprising:

providing a liquid sample to be analyzed;

providing a device of any one of embodiments 12 through 22;

transferring a predetermined volume of the liquid sample to the body of the device;

removing any barrier seal that is present;

applying force to the plunger to urge the liquid sample through the microporous membrane;

removing the microporous membrane from the device;

placing the microporous membrane on a culture medium;

incubating the culture medium;

counting the number of colonies of microorganisms associated with the microporous membrane.

25. A process according to embodiment 23 or embodiment 24 wherein the predetermined volume is transferred to the chamber without the use of an intermediate sample collector.

26. The process according to any one of embodiments 23-25 further comprising adjusting the liquid sample to a predetermined volume after the sample has been transferred into the body of the device.

27. A process, according to any one of embodiments 23-26 wherein the liquid sample comprises a sample of water.

28. A process according to embodiment 27 wherein the sample of water is selected from the group consisting of surface water, water for human or animal consumption, and process water.

29. A process according to embodiment 28 wherein the process water comprises food processing water.

30. A process, according to any one of embodiments 23-29, wherein the culture medium comprises a dry, rehydratable culture medium.

31. A kit for the collection and concentration of samples for microbiological analysis, the kit comprising the device of any one of embodiments 1-22.

32. A kit, according to embodiment 31, further comprising at least one element from the group consisting of gloves, labels, a bag, an intermediate sample collector, a sample collection conduit, a disinfectant, a forceps, a culture medium, a microporous membrane, a prefilter, a culture medium carrier, an incubator, and a reagent.

33. A kit, according to either one of embodiments 31 or 32 wherein at least one of component of the kit is pre-sterilized.

34. A kit, according to embodiment 33 wherein at least one component of the kit is pre-sterilized by irradiation.

35. A device for collecting and concentrating a sample for microbiological analysis, the device comprising:

a body comprising
a cap; and
a chamber wall; and

a plunger comprising
a removable support comprising
   a porous support structure; and
   a drain;

wherein the plunger is dimensioned to provide an essentially watertight fit within the body;

wherein the body, the removable support, and the drain define a flow path for a liquid sample

36. A device according to embodiment 35, further comprising an exit port,

wherein the exit port is in fluid communication with the drain through a passage way outside the plunger.

37. A device according to embodiment 35, further comprising an exit port,

wherein the exit port is in fluid communication with the drain through a passage way in the plunger.

38. A device according to any one of embodiments 35 through 37, further comprising a microporous membrane.

39. A device according to any one of embodiments 35 through 38, wherein the device is configured to hold a predetermined volume of liquid sample that is bounded at least in part by the removable support.

Although the present invention has been described with reference to preferred embodiments and the above-identified figures set forth exemplary embodiments of the present invention, other embodiments are also within the scope of the invention. In all cases, this disclosure presents the invention by way of representation and not limitation.

It should be understood that numerous other modifications and embodiments can be devised by those skilled in the art, which fall within the scope and spirit of the principles of the invention.

Various modifications and alterations to this invention will become apparent to those skilled in the art without departing from the scope and spirit of this invention. It should be understood that this invention is not intended to be unduly limited by the illustrative embodiments and examples set forth herein and that such examples and embodiments are presented by way of example only with the scope of the invention intended to be limited only by the claims set forth herein as follows.

1. A device for collecting and concentrating a sample for microbiological analysis, the device comprising:
a plunger dimensioned to provide an essentially watertight fit within a body;
a removable support configured to position a microporous membrane in a flow path, the support comprising
a porous support structure; and
a drain; and
a body comprising
a chamber wall; and
a base configured to attach to the removable support;
wherein the body, the porous support structure surface, and the drain define a flow path for a liquid sample;
wherein the device is configured to hold a predetermined volume of liquid sample; and
wherein the predetermined volume is bounded at least in part by the removable support configured to position a microporous membrane in a flow path.

2. A device for collecting and concentrating a sample for microbiological analysis, the device comprising:
a plunger dimensioned to provide an essentially watertight fit within a body;
a removable support configured to position a microporous membrane in a flow path, the support comprising
a porous support structure; and
a drain; and
a body comprising
a chamber wall;
a base configured to attach to the removable support;
a plunger orifice; and
a sample intake port;
wherein the sample intake port, the body, the porous support structure surface, and the drain define a flow path for a liquid sample; and
wherein the device is configured to hold a predetermined volume of liquid sample.

3. A device according to claim 1, wherein the removable support further comprising an exit port.

4. A device according to claim 1, wherein the drain further comprises a barrier seal.

5. A device according to claim 2, wherein the predetermined volume is bounded at least in part by the removable support configured to position a microporous membrane in a flow path.

6. A device according to claim 1, wherein the predetermined volume is further bounded by a chamber wall.

7. A device according to claim 1, wherein the predetermined volume is defined by a fill line indicium.

8. A device according to claim 2, wherein the sample intake port is positioned on a region of the body proximate the removable support.

9. A device according to claim 2, wherein the sample intake port is positioned on a region of the body apart from the removable support.

10. A device according to claim 2 wherein the sample intake port further comprises a valve.

11. A device according to claim 10, wherein the valve is actuated by the plunger.

12. A device according to claim 1, the device further comprising a microporous membrane positioned in a liquid flow path.

13. A device according to claim 1, the device further comprising a prefilter.

14. A device according to claim 1, wherein the body comprises at least one fill line indicium.

15. A device according to claim 2, wherein the plunger comprises at least one fill line indicium.

16. A device according to claim 14, wherein the at least one fill line indicium defines a predetermined volume in the device.

17. A device according to claim 1 wherein the exit port comprises a barrier seal.

18-19. (canceled)

20. A device according to claim 1 wherein the body further comprises a gasket seal.

21. A device of claim 1 wherein the device is pre-sterilized.

22. (canceled)

23. A process for collecting and concentrating a sample for microbiological analysis, the process comprising:
providing a liquid sample to be analyzed;
providing the device of claim 12;
transferring a predetermined volume of the liquid sample to the body of the device; and
applying force to the plunger to urge the liquid sample through the microporous membrane.

24. (canceled)

25. A process according to claim 23 wherein the predetermined volume is transferred to the chamber without the use of an intermediate sample collector.

26. The process according to claim 23 further comprising adjusting the liquid sample to a predetermined volume after the sample has been transferred into the body of the device.

27. A process, according to claim 23 wherein the liquid sample comprises a sample of water.

28. A process according to claim 27 wherein the sample of water is selected from the group consisting of surface water, water for human or animal consumption, and process water.

29. (canceled)

30. A process, according to claim 23, wherein the culture medium comprises a dry, rehydratable culture medium.

31. A kit for the collection and concentration of samples for microbiological analysis, the kit comprising the device of claim 1.

32. A kit, according to claim 31, further comprising at least one element from the group consisting of gloves, labels, a bag, an intermediate sample collector, a sample collection conduit, a disinfectant, a forceps, a culture medium, a microporous membrane, a prefilter, a culture medium carrier, an incubator, and a reagent.

33. A kit, according to claim 31 wherein at least one of component of the kit is pre-sterilized.

34. (canceled)

35. A device for collecting and concentrating a sample for microbiological analysis, the device comprising:
a body comprising
a cap; and
a chamber wall; and
a plunger comprising
a removable support comprising
a porous support structure; and
a drain;
wherein the plunger is dimensioned to provide an essentially watertight fit within the body;
wherein the body, the removable support, and the drain define a flow path for a liquid sample

36. A device according to claim 35, further comprising a microporous membrane.
